(19) United States
(12) Patent Application Publication
(10) Pub. No.: US 2025/0140387 A1
(43) Pub. Date: May 1, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yohei MOMOKI, Tokyo (JP)

(21) Appl. No.: 18/925,024

(22) Filed: Oct. 24, 2024

(30) Foreign Application Priority Data
Oct. 27, 2023 (JP) 2023-185034

(51) Int. Cl. G16H 30/40; G06V 10/25; G06V 10/74; G16H 50/20

(52) U.S. Cl. CPC G16H 30/40; G06V 10/25; G06V 10/761; G16H 50/20

(57) ABSTRACT

An information processing apparatus including a processor, wherein the processor is configured to: acquire a comment on findings including a plurality of pieces of element information about at least one region of interest; make a first determination as to whether the comment on findings is described for a plurality of regions of interest or for one region of interest based on the element information; and determine a method for presenting the comment on findings based on a result of the first determination.

### CROSS-REFERENCE TO RELATED APPLICATIONS

This application claims priority from Japanese Application No. 2023-185034, filed on Oct. 27, 2023, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. A creator of an interpretation report, such as a radiologist, interprets medical images and creates an interpretation report including a comment on findings.

In order to support the creation of an interpretation report, a method has been proposed that searches for and presents an appropriate comment on finding from a created past comment-on-findings group. For example, JP2008-146220A discloses a method for executing a process of searching for a structured document database based on word-based input information by a user and executing a process of generating a sentence describing a morphological description of an image based on the input information.

In addition, for example, JP2006-155002A discloses a method for searching for and outputting a reference interpretation report from among a plurality of interpretation reports created at the time of past interpretation and expressed in a structured tag format, using input tag information as a search key.

A new comment on findings may be created by rewriting a part of a past comment on findings obtained as a search result. In order to improve the efficiency of creating a comment on findings in this case, it is desirable to present, as a search result, a comment on findings that requires fewer portions to be rewritten.

### SUMMARY

The present disclosure provides an information processing apparatus, an information processing method, and an information processing program that can support creation of an interpretation report.

According to a first aspect of the present disclosure, there is provided an information processing apparatus comprising at least one processor, in which the processor is configured to: acquire a comment on findings including a plurality of pieces of element information about at least one region of interest; make a first determination as to whether the comment on findings is described for a plurality of regions of interest or for one region of interest based on the element information; and determine a method for presenting the comment on findings based on a result of the first determination.

In the first aspect, the processor may be configured to, in a case of presenting at least one of a plurality of the comments on findings, lower a presentation priority of the comment on findings that is determined to be described for the plurality of regions of interest.

In the first aspect, the processor may be configured to: make a second determination as to whether the comment on findings is described for a plurality of types of the regions of interest or for one type of the plurality of regions of interest, the comment on findings being determined to be described for the plurality of regions of interest in the first determination; and determine the method for presenting the comment on findings based on a result of the second determination.

In the first aspect, the processor may be configured to, in a case of presenting at least one of a plurality of the comments on findings, lower a presentation priority of the comment on findings that is determined to be described for the plurality of types of the plurality of regions of interest.

In the first aspect, the processor may be configured to, in a case of presenting at least one of a plurality of the comments on findings, increase a presentation priority of the comment on findings described for the regions of interest of which a number and a type match a number and a type of new regions of interest for which a new comment on findings is to be created.

In the first aspect, the processor may be configured to make the first determination based on the number of pieces of element information indicating types of the regions of interest included in the comment on findings.

In the first aspect, the processor may be configured to, in a case in which the element information is divided into groups for each region of interest, make the first determination based on the number of groups.

In the first aspect, the comment on findings may consist of a plurality of partial sentences, and the processor may be configured to: make the first determination for each of the partial sentences; and determine a presentation method in a case in which the comment on findings is divided into the partial sentences and presented based on the result of the first determination.

In the first aspect, the processor may be configured to: classify the element information included in the partial sentences into whether the element information is about an attention region of interest that is the region of interest being attended, whether the element information is about a non-attention region of interest that is the region of interest other than the attention region of interest, or whether the element information is common to the attention region of interest and the non-attention region of interest; and determine that the partial sentence including the element information about the non-attention region of interest is described for the plurality of regions of interest.

In the first aspect, the processor may be configured to: acquire new element information about one new region of interest for which a new comment on findings is to be created; and rewrite the comment on findings that is determined to be described for the one region of interest in the first determination in accordance with the new element information and present the rewritten comment on findings.

In the first aspect, the processor may be configured to: acquire a plurality of pieces of new element information about each of a plurality of new regions of interest for which a new comment on findings is to be created; and rewrite the comment on findings that is determined to be described for the one region of interest in the first determination in accordance with the plurality of pieces of new element information and present the rewritten comment on findings.

In the first aspect, the processor may be configured to: acquire a plurality of pieces of new element information about each of a plurality of new regions of interest for which a new comment on findings is to be created; and rewrite the comment on findings described for the regions of interest of which a number and a type match a number and a type of the plurality of new regions of interest, among the comments on findings that are determined to be described for the plurality of regions of interest in the first determination, in accordance with the plurality of pieces of new element information and present the rewritten comment on findings.

In the first aspect, the new element information used for rewriting may indicate at least one of a position, a measurement value, or the number of the new region of interest.

In the first aspect, the processor may be configured to receive an input of the new element information by a user.

In the first aspect, the processor may be configured to: acquire an image including at least one new region of interest for which a new comment on findings is to be created; and generate the new element information based on the image.

In the first aspect, the element information may include at least one of a property, a position, a measurement value, a number, and a diagnosis of a region of interest, or a phrase expressing a change in the region of interest.

According to a second aspect of the present disclosure, there is provided an information processing method executed by a computer, the method comprising: acquiring a comment on findings including a plurality of pieces of element information about at least one region of interest; making a first determination as to whether the comment on findings is described for a plurality of regions of interest or for one region of interest based on the element information; and determining a method for presenting the comment on findings based on a result of the first determination.

According to a third aspect of the present disclosure, there is provided an information processing program for causing a computer to execute a process comprising: acquiring a comment on findings including a plurality of pieces of element information about at least one region of interest; making a first determination as to whether the comment on findings is described for a plurality of regions of interest or for one region of interest based on the element information; and determining a method for presenting the comment on findings based on a result of the first determination.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is an example of a comment on findings described for one region of interest.

FIG. 7 is an example of a comment on findings described for a plurality of regions of interest.

FIG. 8 is an example of a comment on findings described for a plurality of regions of interest.

FIG. 9 is a diagram showing an example of a screen displayed on a display.

FIG. 10 is a diagram for describing rewriting of a comment on findings.

FIG. 11 is a diagram for describing rewriting of a comment on findings.

FIG. 12 is a diagram for describing rewriting of a comment on findings.

FIG. 13 is a diagram for describing rewriting of a comment on findings.

FIG. 14 is a diagram showing an example of a screen displayed on a display.

FIG. 15 is a flowchart showing an example of information processing.

FIG. 16 is an example of a comment on findings consisting of a plurality of partial sentences.

FIG. 17 is an example of a comment on findings consisting of a plurality of partial sentences.

FIG. 18 is an example of a comment on findings consisting of a plurality of partial sentences.

### DETAILED DESCRIPTION

Hereinafter, embodiments of the present disclosure will be described with reference to the drawings.

### First Embodiment

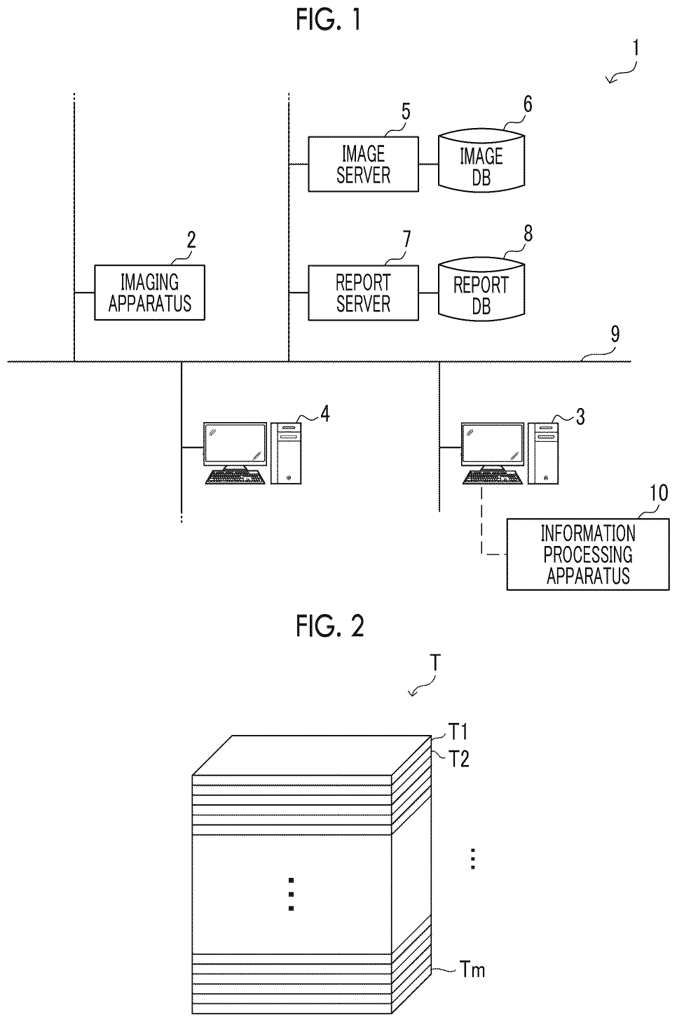

First, a configuration of an information processing system 1 to which an information processing apparatus 10 according to an aspect of the present disclosure is applied will be described with reference to FIG. 1. FIG. 1 is a diagram showing a schematic configuration of the information processing system 1. The information processing system 1 performs imaging of an examination target part of a subject and storing of the captured medical images based on an examination order from a doctor in a medical department using a known ordering system. In addition, the information processing system 1 allows a radiologist to interpret a medical image and create an interpretation report, and allows a doctor of a medical department that is a request source to view the interpretation report.

As shown in FIG. 1, the information processing system 1 includes an imaging apparatus 2, an interpretation workstation (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read-only memory (DVD-ROM) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is stored in the image DB 6.

FIG. 2 is a diagram schematically showing an example of a medical image acquired by the imaging apparatus 2. A medical image T shown in FIG. 2 is, for example, a CT image consisting of a plurality of tomographic images T1 to Tm (m is 2 or more) representing tomographic planes from a head to a waist of one subject (human body). The medical image T is an example of an image of the present disclosure.

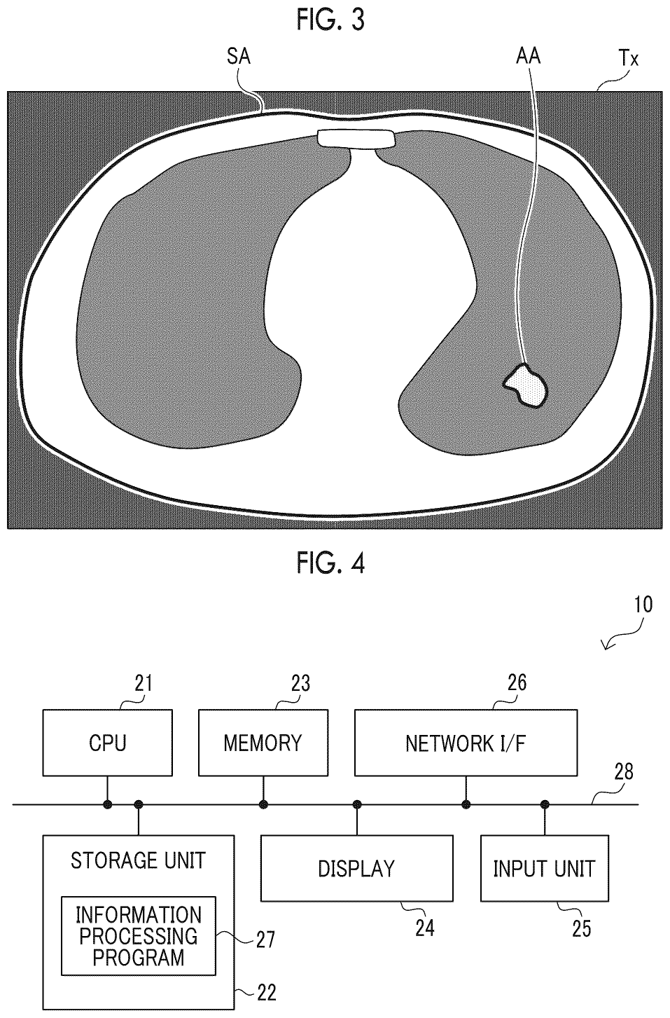

FIG. 3 is a diagram schematically showing an example of one tomographic image Tx out of the plurality of tomographic images T1 to Tm. The tomographic image Tx shown in FIG. 3 represents a tomographic plane including lungs. Each of the tomographic images T1 to Tm may include a region SA of a structure showing various organs and viscera of the human body (for example, lungs, kidneys, and the like), various tissues constituting various organs and viscera (for example, blood vessels, nerves, muscles, and the like), and the like. In addition, each tomographic image may include a lesion region AA such as, for example, nodules, tumors, injuries, defects, and inflammation. In the tomographic image Tx shown in FIG. 3, a lung region is the region SA of the structure, and a nodule region is the lesion region AA. A single tomographic image may include regions SA of a plurality of structures and/or lesion regions AA. The lesion region AA is an example of a region of interest of the present disclosure.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and input devices such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network-attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, an imaging purpose, an imaging date and time, and an imaging location related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the found medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered (details will be described later).

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the found interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Meanwhile, in a case in which the medical image is interpreted and the interpretation report is created in the interpretation WS 3, a past interpretation report (comment on findings) registered in the report DB 8 may be referred to for reference. In this case, a search query corresponding to the medical image to be interpreted is used to search for past comments on findings described in the same or similar findings and/or diagnoses as the medical image to be interpreted. For example, in a case in which a nodule of a lung is included in a medical image to be interpreted, a search is performed using “lung” and “nodule” as a search query, and comments on findings including “lung” and “nodule” are presented as search results.

In addition, a new comment on findings may be created by rewriting a part of a past comment on findings obtained as a search result. For example, a new comment on findings may be completed simply by correcting the position, measurement value, number, and the like of the lesion among descriptions of the lesion included in the past comment on findings. On the other hand, in a case in which the past comment on findings includes descriptions of a plurality of lesions or descriptions of a plurality of different types of lesions, dependencies may be complex, which increases the time and effort required for correction. Therefore, in order to improve the efficiency of creating a comment on findings, it is desirable to present, as a search result, a past comment on findings that requires fewer portions to be rewritten.

Therefore, the information processing apparatus 10 according to the present embodiment supports the creation of interpretation reports by presenting past comments on findings suitable for creating new comments on findings (that is, past comments on findings suitable for reusing). The information processing apparatus 10 will be described below. As described above, the information processing apparatus 10 is encompassed in the interpretation WS 3.

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid-crystal display, an input unit 25 such as a keyboard and a mouse, and a network interface (I/F) 26. The network I/F 26 is connected to the network 9 and performs wired and/or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be applied as appropriate.

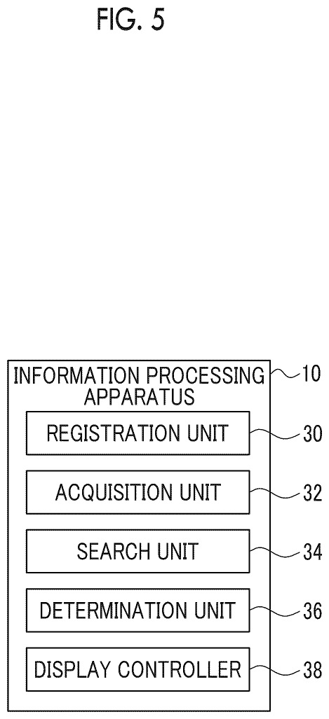

Next, with reference to FIG. 5, an example of a functional configuration of the information processing apparatus 10 according to the present embodiment will be described. As shown in FIG. 5, the information processing apparatus 10 includes a registration unit 30, an acquisition unit 32, a search unit 34, a determination unit 36, and a display controller 38. In a case in which the CPU 21 executes the information processing program 27, the CPU 21 functions as each of the functional units, namely, a registration unit 30, an acquisition unit 32, a search unit 34, a determination unit 36, and a display controller 38.

### Structuring of Interpretation Report

First, a process of structuring an interpretation report will be described as pre-processing executed by the information processing apparatus 10 prior to the process of searching for a comment on findings. As described above, in the report DB 8, created interpretation reports are registered. Specifically, a plurality of comments on findings and diagnostic information and finding information related to each of the comments on findings are registered in the report DB 8 in association with each other. The diagnostic information and the finding information are each an example of element information of the present disclosure.

The comments on findings are registered in the report DB 8 by the report server 7 at the time of creating each interpretation report. The registration unit 30 specifies, each of a plurality of comments on findings (hereinafter referred to as a comment-on-findings group) registered in the report DB 8, element information related to the comments on findings and adds the specified element information to the comments on findings (so-called structuring).

The diagnostic information is, for example, a diagnosis such as an estimated disease name diagnosed based on a medical image. The estimated disease name is an evaluation result estimated based on the lesion included in the medical image, and, for example, the disease name such as “cancer” and “inflammation” and the evaluation result such as “negative/positive”, “benign/malignant”, and “mild/severe” regarding disease names and properties can be mentioned.

In addition, one comment on findings may include a plurality of estimated disease names, such as “renal cell carcinoma is suspected, but angiomyolipoma with a small amount of fatty component may also be possible”. In this case, the registration unit 30 may select one estimated disease name as diagnostic information, and add other estimated disease names that were not selected to the comment on findings as finding information.

The finding information is, for example, information indicating at least one of various findings such as a type (name), a property, a position, a measurement value, and the number of a region of interest, a phrase expressing a change in the region of interest, or a type of diagnostic information other than the diagnostic information to be paired. As described above, the type of diagnostic information other than the diagnostic information to be paired is an estimated disease name or the like that was not selected as diagnostic information.

Examples of the type (name) of the region of interest include the names of structures, such as “lung” and “liver”, and the names of lesions, such as “nodule” and “ground glass opacity”. The property of the region of interest mainly means the features of the lesion. For example, in the case of a nodule, findings indicating opacity such as “solid”, “low opacity”, and “high opacity”, margin shapes such as “well-defined/ill-defined”, “smooth/irregular”, “spicula”, “lobulated”, and “lagged”, and an overall shape such as “round” and “irregular form” can be mentioned. Also, for example, the relationship with the peripheral tissue, such as “protrusion”, and findings regarding the presence or absence of contrast, washout, and the like can be mentioned.

The position of a region of interest means an anatomical position, a position in a medical image, and a relative positional relationship with other regions of interest such as “inside”, “margin”, and “surroundings”. The anatomical position may be indicated by an organ name such as “lung” and “kidney”, and may be expressed in terms of subdivided lungs such as “right lung”, “upper lobe”, and apical segment (“S1”), for example.

The measurement value of a region of interest is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like. In addition, the finding information indicating the measurement value may be divided into predetermined classes (that is, quantized), such as “0 mm or more and less than 5 mm”, “5 mm or more and less than 10 mm”, and “10 mm or more and less than 15 mm”. In this case, for example, in a case in which the comment on findings includes the description “12 mm”, the finding information “10 mm or more and less than 15 mm” is specified. Furthermore, the finding information indicating the measurement value may simply be information indicating whether or not it is described in the comment on findings. This is because measurement values may have a large variation or may not be described.

The number of regions of interest may be expressed as a specific numerical value such as one or two, or may be expressed in relative expressions such as “single/multiple” and “few/many”. A phrase expressing a change in the region of interest is a phrase expressing changes over time in properties, positions, measurement values, numbers, and the like in a case in which follow-up observation for the region of interest is performed, and examples of such phrases include “appearance/disappearance”, “increase/reduction”, “worsening/improvement”, and “metastasis”.

Specifically, the registration unit 30 extracts named entities (words) from the comments on findings registered in the report DB 8, and specifies the element information corresponding to the extracted named entities. As a method for extracting named entities from a comment on findings, a known named entity extraction method using a natural language processing model such as, for example, bidirectional encoder representations from transformers (BERT) can be applied as appropriate.

In addition, the comment-on-findings group may include different words with the same meaning (synonyms), such as “angiomyolipoma” and “AML”. In order to deal with such spelling variations due to synonyms, in a case in which a comment-on-findings group includes synonyms, it is preferable that the registration unit 30 adds the same element information to each of the comments on findings including synonyms (so-called normalization).

Specifically, a dictionary in which a correspondence relationship between named entities that may be included in a comment on findings and element information is defined, in which synonymous named entities are associated with the same element information, may be stored in advance in the storage unit 22. For example, in the dictionary, “angiomyolipoma” and “AML” indicating synonyms may each be associated with the same diagnostic information, “angiomyolipoma”. The registration unit 30 may extract named entities from the comments on findings registered in the report DB 8 and specify the element information included in the comments on findings by referring to a dictionary.

In addition, it is preferable that the registration unit 30 also specifies the factuality of the specified element information. The “factuality” is information indicating whether the finding is found or not, and the degree of certainty thereof and the like. This is because interpretation reports may include not only findings that are clearly found from medical images, but also findings that are not found from medical images, and findings that are suspicious but have a low degree of certainty. For example, since the presence or absence and the degree of fatty components are used to differentiate between AML and renal cell carcinoma (RCC), the interpretation report may deliberately include that “no fatty components are observed”.

In addition, the element information may be information that modifies other element information. In this case, it is preferable that the registration unit 30 also specifies a modification relationship between the pieces of element information. For example, “calcification”, which is an example of the property of the lung nodule, may be described in detail as “microcalcification is observed in the center”. In this case, the registration unit 30 may specify the finding information of “center” and “micro” as other finding information that modifies the finding information of “calcification”. Examples of finding information that modifies “calcification” include “micro”, “coarse”, “scattered”, “center”, “ring-shaped”, and “complete”.

For example, in the case of “renal cell carcinoma”, which is an example of diagnostic information based on the nodule of the kidney, a tissue type such as “clear cell type”, “papillary”, “chromophobe”, and “multilocular cystic” may also be described in the comments on findings. The registration unit 30 may specify element information indicating the tissue type as described above as other element information that modifies the diagnostic information “renal cell carcinoma”.

Furthermore, in a case in which one comment on findings includes element information related to a plurality of regions of interest (lesions), it is preferable that the registration unit 30 specifies element information about each region of interest.

In addition, in a case in which the diagnostic information cannot be specified from the comment on findings, the registration unit 30 may add the diagnostic information “undiagnosed” to the comment on findings. For example, in a case in which a lesion (region of interest) included in a medical image is small and the findings can be interpreted but are below diagnostic criteria predetermined in the guidelines (that is, are not important), only the finding information may be described and not described as diagnostic information. Also, for example, in a case in which the region of interest is clearly benign, only the finding information may be described and not described as diagnostic information. In addition, for example, diagnostic information may not be described due to an oversight by the creator who wrote the comment on findings. Furthermore, for example, there are cases where the registration unit 30 cannot specify diagnostic information from the comment on findings because the sentence structure of the comment on findings is complex.

Furthermore, the registration unit 30 may specify element information based on a medical image related to the comment on findings, such as a medical image registered in the image DB 6 or the like. Specifically, the registration unit 30 may specify element information that is not included in the comment on findings based on the medical image. In addition, the registration unit 30 does not need to specify all of the various types of element information described above, and may specify only a predetermined type of element information (for example, element information indicating the size of the region of interest) based on the medical image.

For example, in a case in which the size of the region of interest is small, it may be less important to describe it or it may not be possible to measure it from the medical image, but in a case in which the size of the region of interest is large, it can be seen from the medical image. Therefore, the description may be omitted from the comment on findings regardless of its size. On the other hand, the size of the region of interest may be related to properties, etc., and even in a case in which the description is omitted from the comment on findings, it may implicitly affect the content of the comment on findings. For example, in lung cancer, the larger the tumor, the more likely it is that internal necrosis will occur. Therefore, it is preferable that the registration unit 30 adds element information that is not included in the comment on findings but can be specified based on the medical image to the comment on findings and registers it in the report DB 8.

An example of a method for specifying element information based on a medical image will be described. The registration unit 30 acquires a medical image related to the comment on findings, and extracts at least one region of interest (for example, a nodule region) included in the medical image. As a method for extracting a region of interest, a method using known computer-aided detection/diagnosis (CAD) technology and artificial intelligence (AI) technology can be applied as appropriate. For example, the registration unit 30 may extract a region of interest from a medical image by using a learning model such as a convolutional neural network (CNN) that has been trained to receive the medical image as an input and extract and output a region of interest included in the medical image.

Then, the registration unit 30 generates element information of the extracted region of interest. For example, the registration unit 30 may generate element information of a region of interest by using a learning model such as a CNN that has been trained in advance to receive the region of interest extracted from the medical image as an input and output the element information of the region of interest. The registration unit 30 adds at least one of pieces of element information generated in this manner to the comment on findings and registers it in the report DB 8.

Furthermore, the registration unit 30 may generate element information based on accessory information attached to a medical image registered in, for example, the image DB 6 or the like. As described above, accessory information is attached to each medical image at the time of being registered in the image DB 6. In addition to this, for example, the registration unit 30 may acquire information included in an examination order and an electronic medical record, information indicating various test results such as a blood test and an infectious disease test, information indicating the result of a health diagnosis, and the like from the external device such as the medical care WS 4, and generate the acquired information as element information.

### Search for Comments on Findings

Next, the process of searching for comments on findings executed by the information processing apparatus 10 will be described.

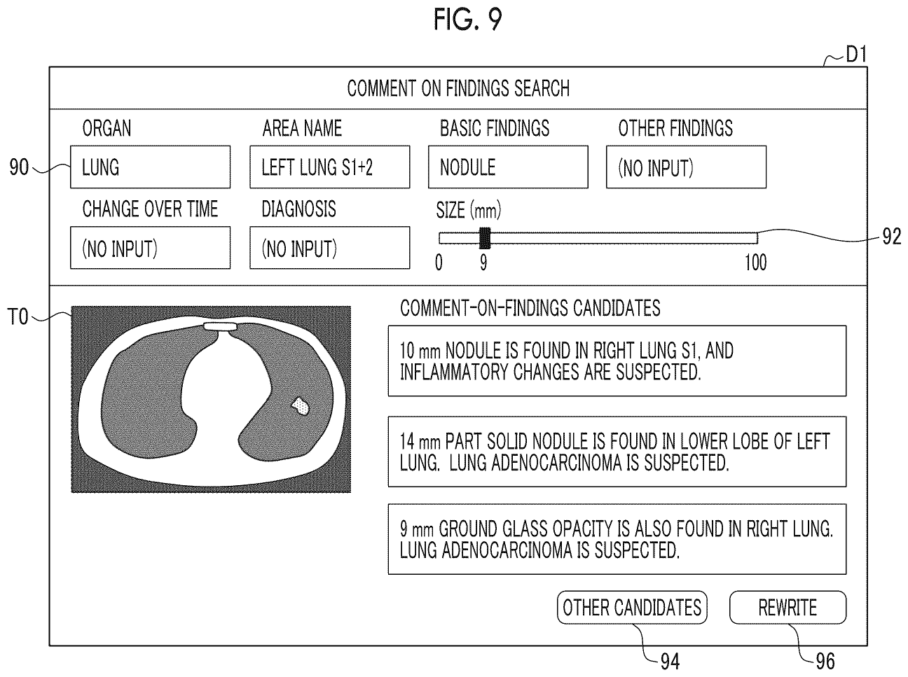

The acquisition unit 32 acquires a search query. For example, the acquisition unit 32 may receive a search query input by the user via the input unit 25 (see FIG. 9). For example, the acquisition unit 32 may acquire a medical image T0 to be interpreted from the image server 5, and generate element information about the medical image T0 as a search query based on the medical image T0. In addition, a search query input by a user and a search query generated based on a medical image may be used in combination.

An example of a method for generating element information (search query) based on a medical image is described. For example, the acquisition unit 32 extracts at least one region of interest (for example, a nodule region) included in the acquired medical image T0. As a method for extracting the region of interest, a known method using a CAD technology, an AI technology, or the like can be appropriately applied. For example, the acquisition unit 32 may extract a region of interest from a medical image by using a learning model such as a CNN that has been trained to receive the medical image as an input and extract and output a region of interest included in the medical image.

The acquisition unit 32 generates element information of the extracted region of interest. For example, the acquisition unit 32 may generate element information of a region of interest by using a learning model such as a CNN that has been trained in advance to receive the region of interest extracted from the medical image as an input and output the element information of the region of interest. The acquisition unit 32 may use at least one of pieces of element information generated in this manner as a search query. In addition, the acquisition unit 32 does not need to specify all of the various types of element information (for example, an organ, an area name, findings, a change over time, and a diagnosis), and may specify only a predetermined type of element information (for example, element information indicating the size of the region of interest) based on the medical image and use the specified element information as a search query.

The search unit 34 searches for a plurality of comment-on-findings candidates related to the search query acquired by the acquisition unit 32 from a comment-on-findings group including a plurality of comments on findings registered in the report DB 8. The search unit 34 may perform a search by giving a priority to a predetermined type of search query. For example, the search unit 34 may perform a search by giving a priority to search queries indicating organs, types, properties, and diagnoses of lesions. On the other hand, the search unit 34 does not need to use a search query indicating the position and measurement values of a lesion for the search. This is because positions and measurement values vary widely from subject to subject, and therefore using them in a search would result in fewer search results. On the other hand, in a case of reusing comment-on-findings candidates, the time and effort required for correcting them is minimal, and therefore it may be more efficient not to use them in a search.

Furthermore, in a case of searching for a comment-on-findings candidate, the search unit 34 may allow ambiguity in the search query used for the search. For example, for a search query indicating a position, comment-on-findings candidates to which finding information indicating a position within a predetermined range (for example, the position of an anatomically adjacent area) is added may be output as search results. Also, for example, for a search query indicating a measurement value, a comment-on-findings candidate to which finding information indicating a measurement value including a difference in a predetermined amount or percentage has been added may be output as a search result. For example, for a search query indicating a diagnosis, comment-on-findings candidates to which diagnostic information indicating different diagnoses belonging to the same classification (for example, “primary lung cancer” and “lung cancer”) is added may be output as search results. In addition, the determination as to whether or not different diagnoses belong to the same classification may be made using, for example, International Statistical Classification of Diseases and Related Health Problems (ICD) codes, dictionaries, ontologies, and the like.

### (Presentation of Comment on Findings)

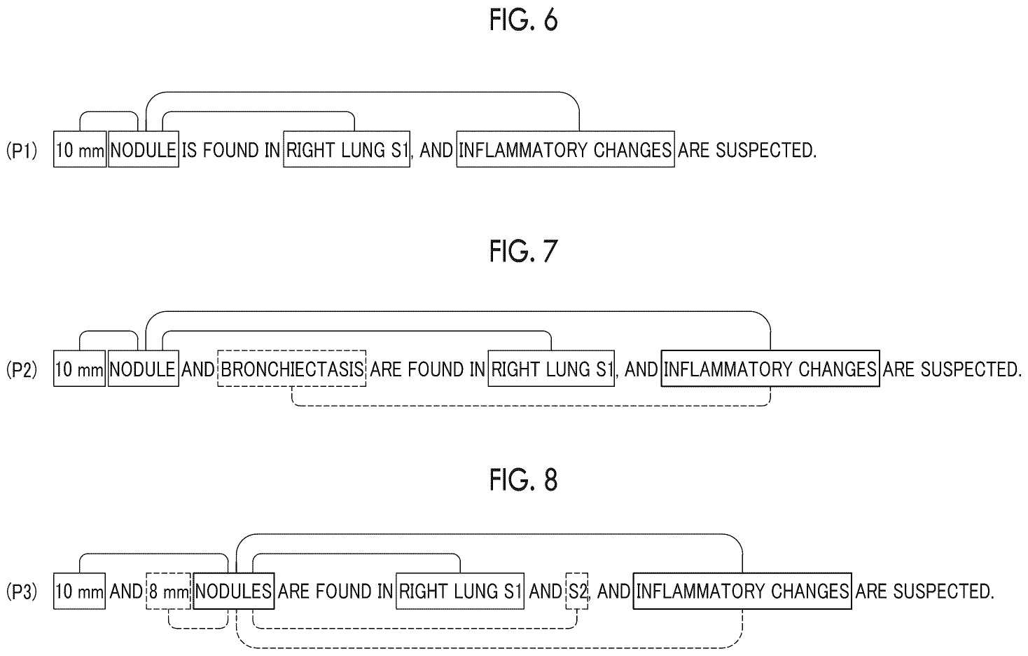

By the above search process, a comment-on-findings candidate corresponding to the search query is acquired. FIGS. 6 to 8 show examples of a plurality of different comment-on-findings candidates P1 to P3 acquired by the acquisition unit 32. A presentation method in a case in which at least one of a plurality of comment-on-findings candidates acquired by the acquisition unit 32 is presented will be described below. The comment-on-findings candidate is an example of a comment on findings of the present disclosure.

Specifically, the acquisition unit 32 acquires, as a search result, a comment-on-findings candidate including a plurality of pieces of element information about at least one region of interest. The determination unit 36 makes a first determination as to whether the comment-on-findings candidate is described for a plurality of regions of interest or for one region of interest based on element information included in the comment-on-findings candidate acquired by the acquisition unit 32. That is, the determination unit 36 makes the first determination by referring to the element information registered in association with each comment-on-findings candidate by the above-mentioned structuring process.

As an example, FIG. 6 shows a comment-on-findings candidate P1 described for one region of interest, and FIGS. 7 and 8 show comment-on-findings candidates P2 and P3 described for a plurality of regions of interest. For the sake of description, FIGS. 6 to 8 also show a graph structure in which element information is enclosed as a node in a rectangle, and pieces of element information (nodes) related to the same region of interest are connected by edges. In FIGS. 7 and 8, in a case in which element information is divided into subgraphs for each region of interest, the structure of one subgraph is shown with a solid line, the structures of the other subgraphs are shown with dotted lines, and element information common to each subgraph is enclosed in a thick solid line rectangle.

Specifically, the determination unit 36 may make the first determination based on the number of pieces of element information indicating types of the regions of interest included in the comment-on-findings candidate. For example, the comment-on-findings candidate P1 includes only “nodule” as element information indicating the type of the region of interest, and is therefore determined to be described for one region of interest. On the other hand, for example, the comment-on-findings candidate P2 includes two items, “nodule” and “bronchiectasis”, as element information indicating the type of the region of interest, and is therefore determined to be described for a plurality of regions of interest.

Further, specifically, the determination unit 36 may make the first determination based on the number of groups in a case in which the element information included in the comment-on-findings candidate is divided into groups for each region of interest. The groups for each region of interest are subgraphs shown in FIGS. 6 to 8. For example, since the comment-on-findings candidate P1 includes only element information related to “nodule” as the region of interest, the number of groups (number of subgraphs) is 1, and it is determined that the comment-on-findings candidate P1 is described for one region of interest.

On the other hand, for example, since the comment-on-findings candidate P2 includes element information related to each of “nodule” and “bronchiectasis” as the regions of interest, the number of groups (number of subgraphs) is 2, and it is determined that the comment-on-findings candidate P2 is described for a plurality of regions of interest. In addition, for example, since the comment-on-findings candidate P3 includes element information related to each of “nodule” in “right lung S1” and “nodule” in “(right lung) S2”, the number of groups (number of subgraphs) is 2, and it is determined that the comment-on-findings candidate P3 is described for a plurality of regions of interest.

The display controller 38 determines a method for presenting the comment-on-findings candidates based on the result of the first determination made by the determination unit 36, and presents at least one comment-on-findings candidate according to the determined presentation method. Specifically, the display controller 38 may lower a presentation priority of a comment-on-findings candidate that is determined to be described for a plurality of regions of interest in the first determination. The display controller 38 may then perform control to display, on the display 24, a predetermined number of comment-on-findings candidates in descending order of priority, or one comment-on-findings candidate with the highest priority, and the like. For example, the display controller 38 may lower a presentation priority of the comment-on-findings candidates P2 and P3 among the comment-on-findings candidates P1 to P3.

In a case of creating a new comment on findings by rewriting a part of a comment-on-findings candidate, a comment-on-findings candidate that requires fewer portions to be rewritten, that is, a comment-on-findings candidate that is described for one region of interest, is considered to be suitable. In other words, it is considered that, in a case in which an attempt is made to create a new comment on findings by rewriting part of a comment-on-findings candidate that is determined to be described for a plurality of regions of interest, it often takes a lot of time and effort to rewrite the comment on findings. Therefore, by lowering the presentation priority of a comment-on-findings candidate that is determined to be described for a plurality of regions of interest, the efficiency of creating a new comment on findings can be improved.

FIG. 9 shows an example of a screen D1 displayed on the display 24 by the display controller 38. At the upper part of the screen D1, a text box 90 for each item for inputting a search query and a slider bar 92 for designating the size of the region of interest are displayed. A user uses the text box 90 and slider bar 92 to input at least one search query. In the example shown in FIG. 9, “lung”, “left lung S1+2”, “nodule”, and “9 mm” are input as the search query. At the lower part of the screen D1, the medical image T0 to be interpreted and three comment-on-findings candidates as a result of a search performed by the search unit 34 using the input search query are displayed. The user checks the presented comment-on-findings candidates, and in a case in which he/she desires to check other comment-on-findings candidates, he/she selects the “other candidates” button 94. In this case, the display controller 38 re-presents other comment-on-findings candidates (that is, comment-on-findings candidates with a lower priority).

On the other hand, in a case in which the user determines that the presented comment-on-findings candidates include a comment-on-findings candidate that can serve as the basis for a new comment on findings, the user selects the “rewrite” button 96. In this case, the display controller 38 rewrites the comment-on-findings candidate and presents the rewritten comment-on-findings candidate again.

### (Rewriting of Comment on Findings)

The process of rewriting a comment-on-findings candidate will be described below.

The acquisition unit 32 acquires new element information about a new region of interest for which a new comment on findings is to be created. Furthermore, in a case in which there are a plurality of new regions of interest for which new comment on findings is to be created, the acquisition unit 32 acquires a plurality of pieces of new element information about each of the new regions of interest. Here, the new element information is information used to rewrite the comments on findings. Generally, in a case of rewriting a comment-on-findings candidate, information that is fundamental to the content of the comment on findings, such as the organ, type of lesion, properties, and diagnosis, is not rewritten and is often reused as it is. Therefore, the new element information used for rewriting may be, for example, information indicating at least one of a position, a measurement value, or the number of the new region of interest.

Specifically, the acquisition unit 32 may set a search query as the new element information. Furthermore, for example, the acquisition unit 32 may receive an input of new element information by the user. Further, for example, the acquisition unit 32 may acquire a medical image T0 including at least one new region of interest for which a new comment on findings is to be created, and generate the new element information based on the medical image T0. The method for generating the new element information based on the medical image T0 is similar to the method for generating element information (search query) based on the medical image described above, for example.

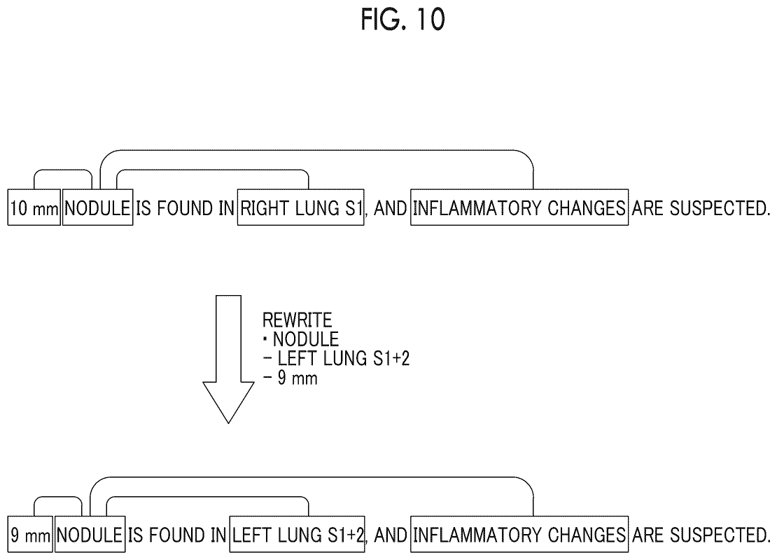

The display controller 38 rewrites the comment-on-findings candidate in accordance with the new element information acquired by the acquisition unit 32 and presents the rewritten comment-on-findings candidate. An example of rewriting a comment-on-findings candidate in accordance with new element information will be described with reference to FIGS. 10 to 13. In the examples shown in FIGS. 10 to 13, the comment-on-findings candidate to be rewritten is the comment-on-findings candidate P1 that is determined to be described for one region of interest in the first determination.

FIG. 10 is an example in which a comment-on-findings candidate described for one region of interest is rewritten in accordance with new element information about one new region of interest for which a new comment on findings is to be created. As new element information, three items have been acquired: “nodule” indicating the type of the new region of interest, “left lung S1+2” indicating its position, and “9 mm” indicating its measurement value (size). In this case, the display controller 38 rewrites the part of the comment-on-findings candidate, “right lung S1”, which indicates the position of the region of interest, to “left lung S1+2”, and rewrites the part, “10 mm”, which indicates the measurement value (size) of the region of interest, to “9 mm”.

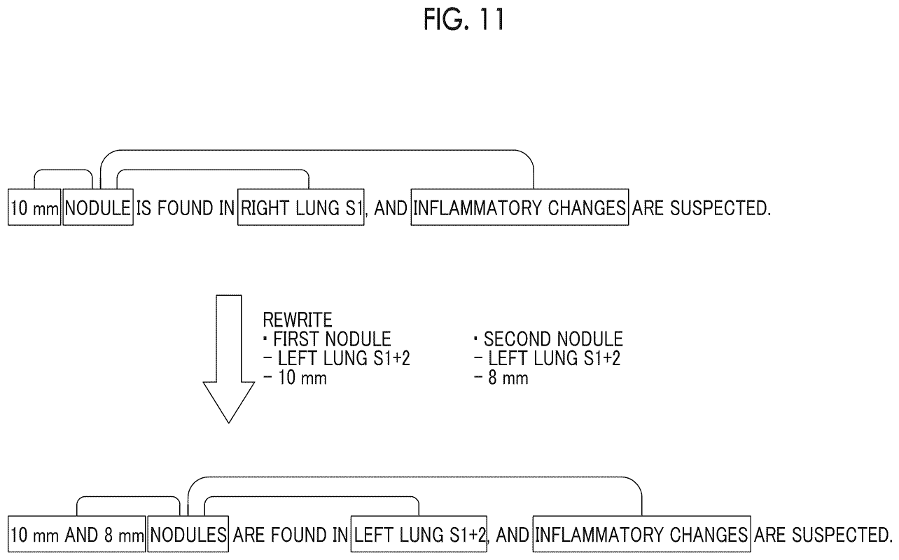

FIGS. 11 to 13 are examples in which a comment-on-findings candidate described for one region of interest is rewritten in accordance with a plurality of pieces of new element information about each of a plurality of new regions of interest for which a new comment on findings is to be created. In the example shown in FIG. 11, the type of the plurality of new regions of interest is common to “nodule” (first nodule, second nodule), their position is common to “left lung S1+2”, and their measurement values (sizes) are different, “10 mm” and “8 mm”, respectively. In this case, the display controller 38 rewrites the part of the comment-on-findings candidate, “right lung S1”, which indicates the position of the region of interest, to the common “left lung S1+2”, and rewrites the part, “10 mm”, which indicates the measurement value (size) of the region of interest, to “10 mm and 8 mm”.

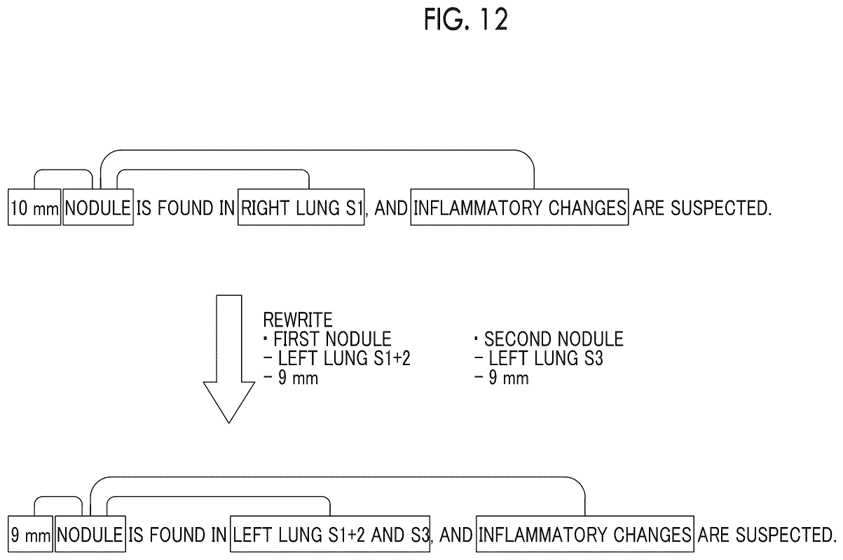

In the example shown in FIG. 12, the type of the plurality of new regions of interest is common to “nodule” (first nodule, second nodule), their positions are different, “left lung S1+2” and “left lung S3”, respectively, and their measurement value (size) is common to “9 mm”. In this case, the display controller 38 rewrites the part of the comment-on-findings candidate, “right lung S1”, which indicates the position of the region of interest, to “left lung S1+2 and S3”, and rewrites the part, “10 mm”, which indicates the measurement value (size) of the region of interest, to the common “9 mm”.

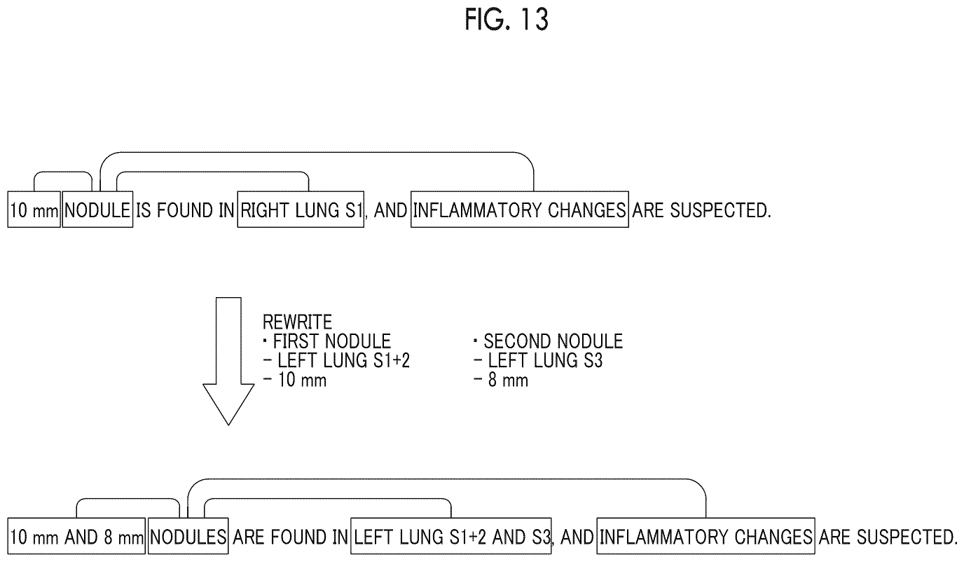

In the example shown in FIG. 13, the type of the plurality of new regions of interest is common to “nodule” (first nodule, second nodule), their positions are different, “left lung S1+2” and “left lung S3”, respectively, and their measurement values (sizes) are different, “10 mm” and “8 mm”, respectively. In this case, the display controller 38 rewrites the part of the comment-on-findings candidate, “right lung S1”, which indicates the position of the region of interest, to “left lung S1+2 and S3”, and rewrites the part, “10 mm”, which indicates the measurement value (size) of the region of interest, to “10 mm and 8 mm”.

The same comment on findings can be generated by performing a similar rewriting based on the comment-on-findings candidates described for the plurality of regions of interest shown in FIG. 8. However, such a comment-on-findings candidate described for a plurality of regions of interest has many portions that need to be rewritten, and thus the processing time required for rewriting may increase.

As shown in the examples shown in FIGS. 10 to 13, a comment-on-findings candidate that is determined to be described for one region of interest in the first determination can be easily rewritten, regardless of whether the new region of interest for which a new comment on findings is to be created is single or multiple. In other words, it can be said that a comment-on-findings candidate described for one region of interest has high general-purpose properties in the case of reusing, and it is preferable to present the comment-on-findings candidate preferentially.

The display controller 38 presents at least one comment-on-findings candidate rewritten in accordance with the new element information. For example, the display controller 38 may perform control such that each of the comment-on-findings candidates presented when the “rewrite” button 96 on the screen D1 is selected is rewritten in accordance with the new element information and then displayed on the display 24.

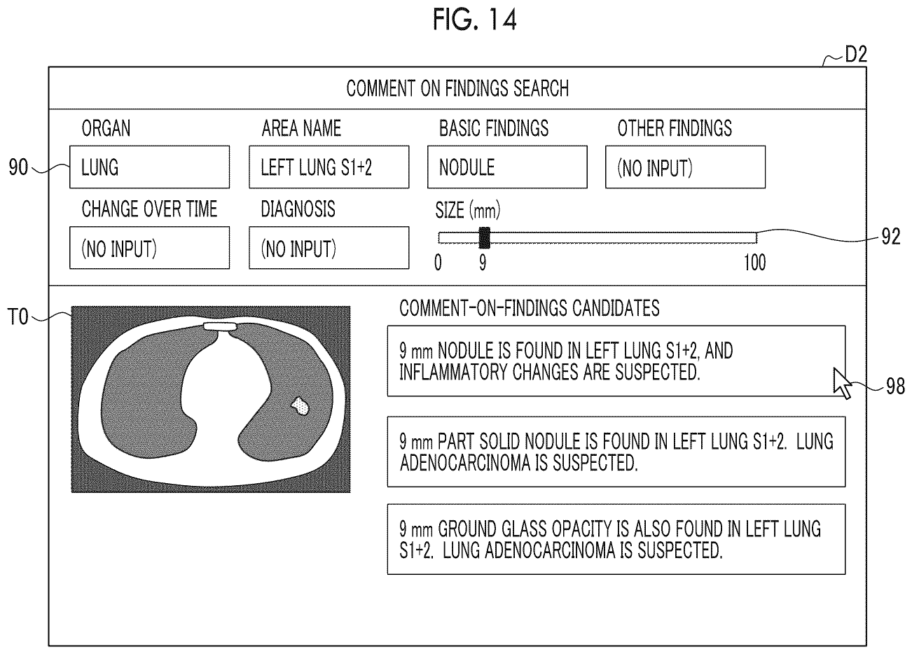

FIG. 14 shows an example of a screen D2 displayed on the display 24 by the display controller 38. The screen D2 is a screen that transitions in a case in which the “rewrite” button 96 on the screen D1 is selected. At the lower part of the screen D2, each of the plurality of comment-on-findings candidates displayed on screen D1 is rewritten in accordance with the search query (new element information) and displayed.

The user checks the presented rewritten comment-on-findings candidates and uses a mouse pointer 98 to select an appropriate one as a comment on findings for the medical image T0 to be interpreted. The display controller 38 registers the selected rewritten comment-on-findings candidate in the report DB 8 as a comment on findings for the medical image T0 to be interpreted. The display controller 38 may receive corrections by the user before registering the rewritten comment-on-findings candidate in the report DB 8.

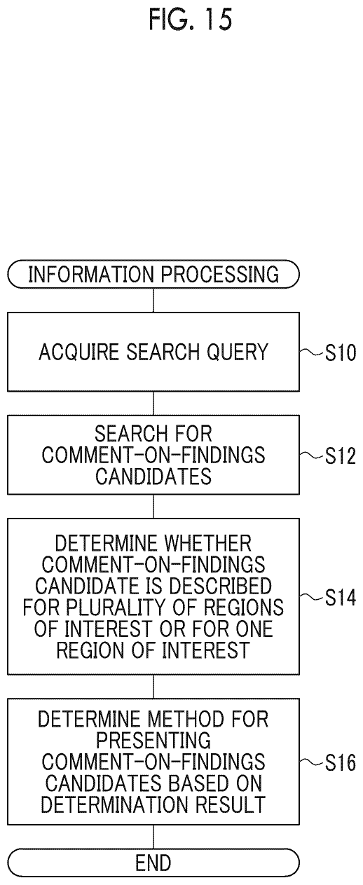

Next, with reference to FIG. 15, operations of the information processing apparatus 10 according to the embodiment will be described. In the information processing apparatus 10, as the CPU 21 executes the information processing program 27, information processing shown in FIG. 15 is executed. The information processing is executed, for example, in a case in which the user provides an instruction to start execution via the input unit 25. It is assumed that the process of structuring the interpretation report described above has been executed in advance.

In Step S10, the acquisition unit 32 acquires a search query. In Step S12, the search unit 34 searches for a plurality of comment-on-findings candidates related to the search query acquired in Step S10 from a comment-on-findings group including a plurality of comments on findings registered in the report DB 8.

In Step S14, the determination unit 36 determines whether the comment-on-findings candidate is described for a plurality of regions of interest or for one region of interest based on element information included in the comment-on-findings candidate searched for (acquired) in Step S12 (first determination). In Step S16, the display controller 38 determines a method for presenting the comment-on-findings candidates based on the result of the first determination in Step S14, and performs control to display, on the display 24, a screen presenting at least one comment-on-findings candidate according to the determined presentation method. In a case in which Step S16 is completed, the present information processing ends.

As described above, the information processing apparatus 10 according to the present embodiment comprises at least one processor. The processor acquires a comment on findings including a plurality of pieces of element information about at least one region of interest, makes a first determination as to whether the comment on findings is described for a plurality of regions of interest or for one region of interest based on the element information, and determines a method for presenting the comment on findings based on a result of the first determination.

That is, with the information processing apparatus 10 according to the present embodiment, in a case in which an attempt is made to create a new comment on findings by rewriting a part of the comment-on-findings candidate acquired as the search result, the comment-on-findings candidate described for one region of interest can be preferentially presented. Since a comment-on-findings candidate described for one region of interest requires fewer portions to be rewritten, the efficiency of creating a comment on findings can be improved by preferentially presenting such a comment-on-findings candidate. Therefore, it is possible to support the creation of an interpretation report.

### Second Embodiment

For example, for a lesion with a low frequency of occurrence and a lesion with a high frequency of occurrence that is in conjunction with another lesion, there may be few past comments on findings described only for that lesion. In this case, even in a case in which an attempt is made to preferentially present the comment on findings described for one region of interest as in the first embodiment, there may be no appropriate comments on findings or the number of comments on findings may be small.

Therefore, the information processing apparatus 10 according to the present embodiment supports the creation of an interpretation report by presenting, among the comment-on-findings candidates acquired as search results, parts suitable for creating a new comment on findings. That is, in the present embodiment, even in a case in which a comment on findings is described for a plurality of regions of interest, the part suitable for reusing is preferentially presented. Hereinafter, functions of the information processing apparatus 10 according to the present embodiment will be described, but descriptions that overlap with those of the first embodiment will be omitted.

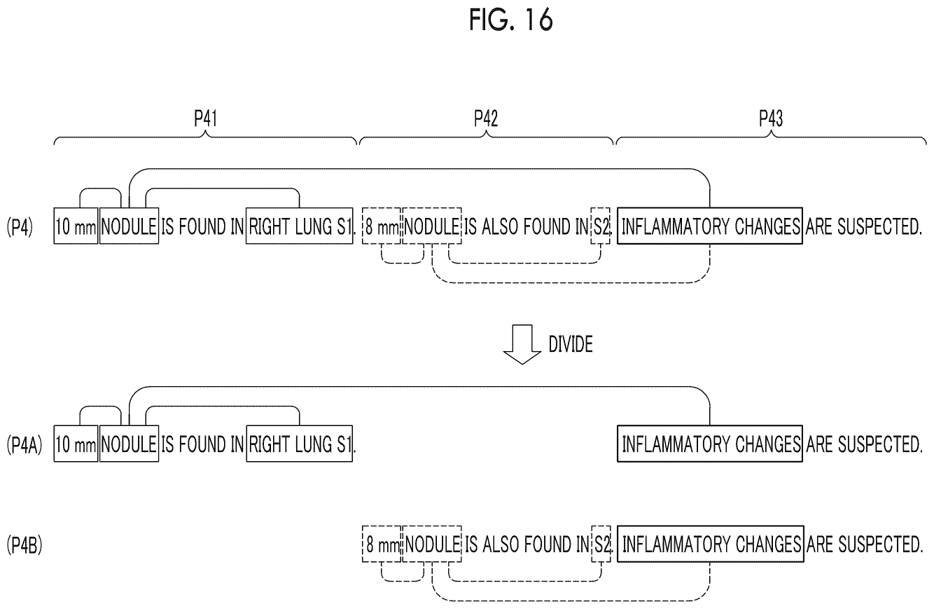

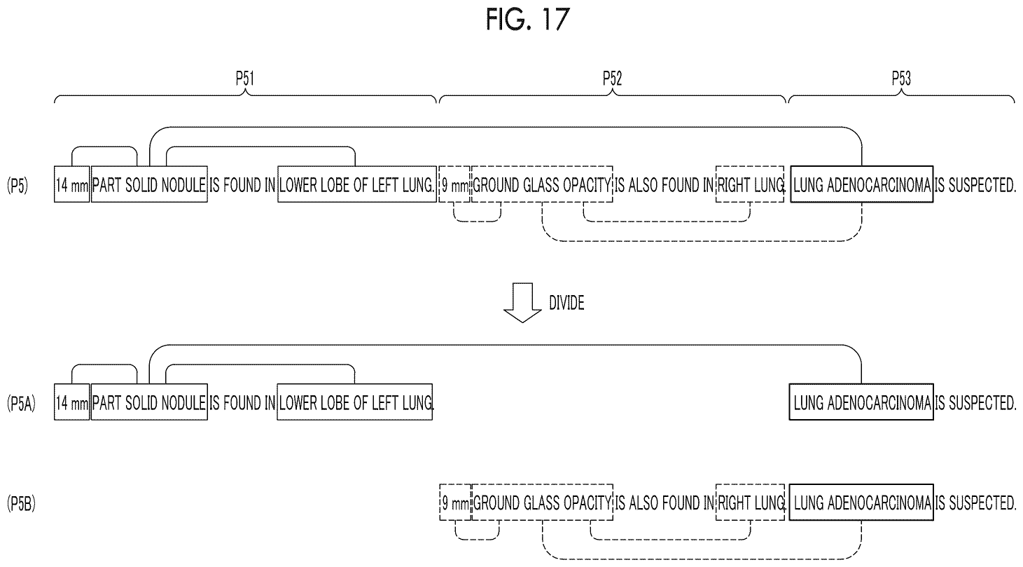

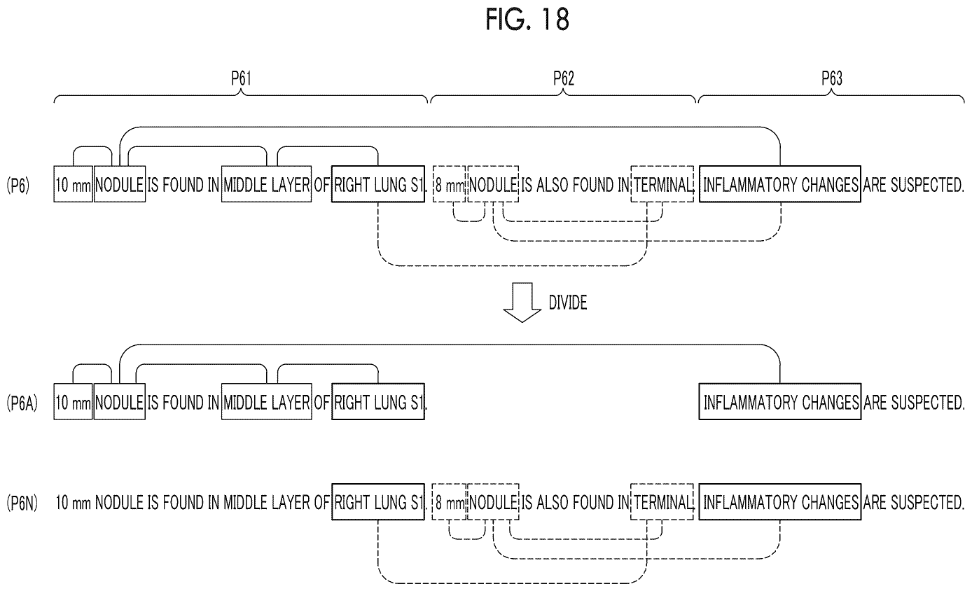

The acquisition unit 32 acquires the comment-on-findings candidates searched for by the search unit 34 in response to the search query. FIGS. 16 to 18 show examples of a plurality of different comment-on-findings candidates P4 to P6 acquired by the acquisition unit 32. For the sake of description, FIGS. 16 to 18 also show a graph structure, similarly to FIG. 6 and the like.

As shown in FIGS. 16 to 18, a comment-on-findings candidate may consist of a plurality of partial sentences. The partial sentence referred to here is one sentence separated by a period, and is, for example, partial sentence P41 in FIG. 16 (“10 mm nodule is found in the right lung S1”.). The comment-on-findings candidate is a set of partial sentences, and is, for example, a sentence consisting of three sentences in FIG. 16: partial sentence P41 (“10 mm nodule is found in the right lung S1”.), partial sentence P42 (“8 mm nodule is also found in S2”.), and partial sentence P43 (“Inflammatory changes are suspected”.). The comment-on-findings candidate P4 consists of partial sentences P41 to P43. The comment-on-findings candidate P5 consists of partial sentences P51 to P53. The comment-on-findings candidate P6 consists of partial sentences P61 to P63.

The determination unit 36 divides the comment-on-findings candidates acquired by the acquisition unit 32 into partial sentences, and makes a first determination for each partial sentence. That is, the determination unit 36 makes a first determination as to whether the partial sentence is described for a plurality of regions of interest or for one region of interest based on element information included in the partial sentence.

Specifically, the determination unit 36 classifies the element information included in the partial sentences into whether the element information is about an attention region of interest that is the region of interest being attended, whether the element information is about a non-attention region of interest that is the region of interest other than the attention region of interest, or whether the element information is common to the attention region of interest and the non-attention region of interest. Then, the determination unit 36 determines that the partial sentence including the element information about the non-attention region of interest is described for the plurality of regions of interest.

The display controller 38 determines a presentation method in a case in which the comment-on-findings candidate is divided into partial sentences and presented based on the result of the first determination made by the determination unit 36, and presents at least one partial sentence according to the determined presentation method. Specifically, the display controller 38 may exclude partial sentences that are determined to be described for a plurality of regions of interest in the first determination (that is, partial sentences that include element information about non-attention regions of interest) and control the other partial sentences to be displayed on the display 24. The other partial sentences are, in other words, partial sentences that include element information about the attention region of interest and partial sentences that include element information common to the attention region of interest and the non-attention region of interest (hereinafter simply referred to as “common partial sentences”).

For example, in the comment-on-findings candidate P4, the first “nodule” is set as an attention region of interest and the second “nodule” is set as a non-attention region of interest. Since the partial sentence P41 includes only element information related to the first “nodule”, it is determined to be a partial sentence related to an attention region of interest. Since the partial sentence P42 includes only element information related to the second “nodule”, it is determined to be a partial sentence related to a non-attention region of interest. Since the partial sentence P43 includes only element information common to the first “nodule” and the second “nodule”, it is determined to be a common partial sentence. The display controller 38 excludes the partial sentence P42 and presents the sentence including the partial sentence P41 and the partial sentence P43 as a comment-on-findings candidate P4A.

On the other hand, in a case in which the first “nodule” is determined as a non-attention region of interest and the second “nodule” is determined as an attention region of interest, the determination of the partial sentence P41 and the partial sentence P42 will be reversed. The display controller 38 excludes the partial sentence P41 and presents the sentence including the partial sentence P42 and the partial sentence P43 as a comment-on-findings candidate P4B.

Further, for example, in the comment-on-findings candidate P5, the “part solid nodule” is set as an attention region of interest and the “ground glass opacity” is set as a non-attention region of interest. Since the partial sentence P51 includes only element information related to the “part solid nodule”, it is determined to be a partial sentence related to an attention region of interest. Since the partial sentence P52 includes only element information related to the “ground glass opacity”, it is determined to be a partial sentence related to a non-attention region of interest. Since the partial sentence P53 includes only element information common to the “part solid nodule” and the “ground glass opacity”, it is determined to be a common partial sentence. The display controller 38 excludes the partial sentence P52 and presents the sentence including the partial sentence P51 and the partial sentence P53 as a comment-on-findings candidate P5A.

On the other hand, in a case in which the “part solid nodule” is determined as a non-attention region of interest and the “ground glass opacity” is determined as an attention region of interest, the determination of the partial sentence P51 and the partial sentence P52 will be reversed. The display controller 38 excludes the partial sentence P51 and presents the sentence including the partial sentence P52 and the partial sentence P53 as a comment-on-findings candidate P5B.

Further, for example, in the comment-on-findings candidate P6, the first “nodule” is set as an attention region of interest and the second “nodule” is set as a non-attention region of interest. Since the partial sentence P61 includes element information related to the first “nodule” and element information common to the first “nodule” and the second “nodule” (“right lung S1”), it is determined to be a common partial sentence. Since the partial sentence P62 includes only element information related to the second “nodule”, it is determined to be a partial sentence related to a non-attention region of interest. Since the partial sentence P63 includes element information common to the first “nodule” and the second “nodule”, it is determined to be a common partial sentence. The display controller 38 excludes the partial sentence P62 and presents the sentence including the partial sentence P61 and the partial sentence P63 as a comment-on-findings candidate P6A.

On the other hand, in a case in which the first “nodule” is determined as a non-attention region of interest and the second “nodule” is determined as an attention region of interest, there will be no partial sentences that can be divided and presented from the comment-on-findings candidate P6. In this case, since the partial sentence P61 includes element information related to the first “nodule”, it is determined to be a partial sentence related to a non-attention region of interest. Therefore, the partial sentence P61 needs to be excluded in the case of being presented as a comment-on-findings candidate; however, because it includes element information (“right lung S1”) that is common to the first “nodule” and the second “nodule”, excluding the partial sentence P61 would make the meaning of the partial sentence P62 incomprehensible. For example, in a case in which the partial sentence P61 is excluded, it is unclear what the “terminal” at the beginning of the partial sentence P62 refers to. Furthermore, for example, in a case in which the partial sentence P61 is left as it is, element information related only to the non-attention region of interest (“middle layer”, “10 mm”, “nodule”) will remain, which will require time and effort to correct at the time of rewriting. Therefore, the display controller 38 does not present such a comment-on-findings candidate P6N.

As described above, the information processing apparatus 10 according to the present embodiment makes the first determination for each of a plurality of partial sentences included in the comment on findings, and determines a presentation method in a case in which the comment on findings is divided into the partial sentences and presented based on the result of the first determination. That is, with the information processing apparatus 10 according to the present embodiment, since it is possible to present more comment-on-findings candidates described for one region of interest, it is possible to improve the efficiency of creating a comment on findings. Therefore, it is possible to support the creation of an interpretation report.

### Third Embodiment

In each of the above embodiments, a form has been described in which a comment on findings described for one region of interest (that is, a comment on findings with high general-purpose properties) is presented with a priority, with an emphasis on the efficiency of creating the comment on findings, but the present disclosure is not limited to the above. For example, in a case in which there are a plurality of new regions of interest for which a new comment on findings is to be created, it may be possible to create more accurate comments on findings by presenting past comments on findings described for the same number and types of regions of interest as the new regions of interest.

Therefore, the information processing apparatus 10 according to the present embodiment supports the creation of an interpretation report by presenting appropriate comment-on-findings candidates according to a new region of interest for which a new comment on findings is to be created. That is, in the present embodiment, even in a case in which a comment on findings is described for a plurality of regions of interest, a comment on findings that is suitable for a new region of interest is preferentially presented. Hereinafter, functions of the information processing apparatus 10 according to the present embodiment will be described, but descriptions that overlap with those of the above embodiments will be omitted.

The acquisition unit 32 acquires the comment-on-findings candidates searched for by the search unit 34 in response to the search query. In addition, the acquisition unit 32 acquires new element information about a new region of interest for which a new comment on findings is to be created. Note that, in a case in which there are a plurality of new regions of interest for which new comment on findings is to be created, the acquisition unit 32 acquires a plurality of pieces of new element information about each of the new regions of interest.

The determination unit 36 makes a first determination on the comment-on-findings candidate acquired by the acquisition unit 32. Accordingly, it is determined whether the comment-on-findings candidate is described for a plurality of regions of interest or for one region of interest. For example, as described above, the comment-on-findings candidates P2 and P3 shown in FIGS. 7 and 8 are determined to be described for a plurality of regions of interest.

Then, the determination unit 36 makes a second determination as to whether the comment-on-findings candidate is described for a plurality of types of the regions of interest or for one type of the plurality of regions of interest, the comment-on-findings candidate being determined to be described for the plurality of regions of interest in the first determination. In this case, it is preferable that the determination unit 36 specifies how many types of regions of interest are described in the comment-on-findings candidates, which are determined to be described for the plurality of types of regions of interest.

For example, the comment-on-findings candidate P2 includes element information indicating two types of regions of interest, namely, “nodule” and “bronchiectasis”, and is therefore determined to be described for two types of regions of interest. Furthermore, for example, the comment-on-findings candidate P3 includes element information indicating two regions of interest of the same type, namely, “nodule” in “right lung S1” and “nodule” in “(right lung) S2”, and is therefore determined to be described for one type of a plurality of regions of interest.

The display controller 38 determines a method for presenting the comment-on-findings candidates based on the result of the second determination made by the determination unit 36, and presents at least one comment-on-findings candidate according to the determined presentation method. Specifically, the display controller 38 may increase the presentation priority of a comment-on-findings candidate described for regions of interest of which a number and a type match the number and type of new regions of interest. For example, in a case in which the new regions of interest are two types of two regions of interest, the priority of the comment-on-findings candidate P2 of which a number and a type match may be increased. Further, for example, in a case in which the new regions of interest are two regions of interest of the same type, the priority of the comment-on-findings candidate P3 of which a number and a type match may be increased.

In addition, the display controller 38 may rewrite the comment-on-findings candidate in accordance with the new element information acquired by the acquisition unit 32 and present the rewritten comment-on-findings candidate. That is, the display controller 38 may rewrite the comment-on-findings candidate described for the regions of interest of which a number and a type match a number and a type of the plurality of new regions of interest, among the comment-on-findings candidates that are determined to be described for the plurality of regions of interest in the first determination, in accordance with the plurality of pieces of new element information and present the rewritten comment-on-findings candidate.

As described above, the information processing apparatus 10 according to the present embodiment makes a second determination as to whether the comment on findings is described for a plurality of types of the regions of interest or for one type of the plurality of regions of interest, the comment on findings being determined to be described for the plurality of regions of interest in the first determination, and determines a method for presenting the comment on findings based on a result of the second determination. That is, with the information processing apparatus 10 according to the present embodiment, it is possible to present comment-on-findings candidates described for the same number and types of regions of interest as the new regions of interest, thereby enabling accurate comments on findings to be created and supporting the creation of interpretation reports.

In the third embodiment, a form has been described in which comment-on-findings candidates described for the same number and types of regions of interest as new regions of interest are preferentially presented, but the present disclosure is not limited to thereto. For example, the display controller 38 may lower the presentation priority of a comment-on-findings candidate that is determined to be described for a plurality of types of regions of interest in the second determination. That is, regardless of the number and types of new regions of interest, the priority of a comment-on-findings candidate described for a plurality of types of regions of interest may be lowered. This is because, in general, a comment on findings described for a plurality of types of regions of interest has more complex dependencies than a comment on findings described for one type of a plurality of regions of interest, and may be difficult to rewrite.

Note that, in each of the above embodiments, a form assuming interpretation for medical images has been described, but the present disclosure is not limited thereto. The information processing apparatus 10 according to one aspect of the present disclosure can be applied to various images including a region of interest, which are obtained by imaging a subject. For example, the information processing apparatus 10 may be applied to an image acquired using an apparatus, a building, a pipe, a welded portion, or the like as a subject in a non-destructive examination such as a radiation transmission examination and an ultrasonic flaw detection examination. In this case, for example, the region of interest indicates cracks, flaws, bubbles, foreign matter, or the like.

In addition, in each of the above embodiments, for example, as hardware structures of processing units that execute various kinds of processing, such as the registration unit 30, the acquisition unit 32, the search unit 34, the determination unit 36, and the display controller 38, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiments, the various programs are described as being stored (installed) in the storage unit in advance; however, the present disclosure is not limited thereto. The various programs may be provided in a form recorded in a recording medium such as a compact disc read-only memory (CD-ROM), a digital versatile disc read-only memory (DVD-ROM), and a Universal Serial Bus (USB) memory. In addition, the various programs may be configured to be downloaded from an external device via a network. Furthermore, the technology of the present disclosure extends to a storage medium that non-transitorily stores a program, in addition to the program.

The technology of the present disclosure can be appropriately combined with the above embodiment and examples. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, it goes without saying that unnecessary parts may be deleted, new elements may be added, or replacements may be made to the described contents and illustrated contents shown above within a range that does not deviate from the gist of the technology of the present disclosure.

Regarding the embodiments, the following supplementary notes are further disclosed.

### Supplementary Note 1

An information processing apparatus comprising at least one processor,
in which the processor is configured to:
   acquire a comment on findings including a plurality of pieces of element information about at least one region of interest;
   make a first determination as to whether the comment on findings is described for a plurality of regions of interest or for one region of interest based on the element information; and
   determine a method for presenting the comment on findings based on a result of the first determination.

### Supplementary Note 2

The information processing apparatus according to Supplementary Note 1,
in which the processor is configured to, in a case of presenting at least one of a plurality of the comments on findings, lower a presentation priority of the comment on findings that is determined to be described for the plurality of regions of interest.

### Supplementary Note 3

The information processing apparatus according to Supplementary Note 1 or 2,
in which the processor is configured to:
   make a second determination as to whether the comment on findings is described for a plurality of types of the regions of interest or for one type of the plurality of regions of interest, the comment on findings being determined to be described for the plurality of regions of interest in the first determination; and
   determine the method for presenting the comment on findings based on a result of the second determination.

### Supplementary Note 4

The information processing apparatus according to Supplementary Note 3,
in which the processor is configured to, in a case of presenting at least one of a plurality of the comments on findings, lower a presentation priority of the comment on findings that is determined to be described for the plurality of types of the plurality of regions of interest.

### Supplementary Note 5

The information processing apparatus according to Supplementary Note 3 or 4,
in which the processor is configured to, in a case of presenting at least one of a plurality of the comments on findings, increase a presentation priority of the comment on findings described for the regions of interest of which a number and a type match a number and a type of new regions of interest for which a new comment on findings is to be created.

### Supplementary Note 6

The information processing apparatus according to any one of Supplementary Notes 1 to 5,
in which the processor is configured to make the first determination based on the number of pieces of element information indicating types of the regions of interest included in the comment on findings.

### Supplementary Note 7

The information processing apparatus according to any one of Supplementary Notes 1 to 6,
in which the processor is configured to, in a case in which the element information is divided into groups for each region of interest, make the first determination based on the number of groups.

### Supplementary Note 8

The information processing apparatus according to any one of Supplementary Notes 1 to 7,
in which the comment on findings consists of a plurality of partial sentences, and
the processor is configured to:
   make the first determination for each of the partial sentences; and
   determine a presentation method in a case in which the comment on findings is divided into the partial sentences and presented based on the result of the first determination.

### Supplementary Note 9

The information processing apparatus according to Supplementary Note 8,
in which the processor is configured to:
   classify the element information included in the partial sentences into whether the element information is about an attention region of interest that is the region of interest being attended, whether the element information is about a non-attention region of interest that is the region of interest other than the attention region of interest, or whether the element information is common to the attention region of interest and the non-attention region of interest; and
   determine that the partial sentence including the element information about the non-attention region of interest is described for the plurality of regions of interest.

### Supplementary Note 10

The information processing apparatus according to any one of Supplementary Notes 1 to 9,
in which the processor is configured to:
   acquire new element information about one new region of interest for which a new comment on findings is to be created; and
   rewrite the comment on findings that is determined to be described for the one region of interest in the first determination in accordance with the new element information and present the rewritten comment on findings.

### Supplementary Note 11

The information processing apparatus according to any one of Supplementary Notes 1 to 10,
in which the processor is configured to:
   acquire a plurality of pieces of new element information about each of a plurality of new regions of interest for which a new comment on findings is to be created; and
   rewrite the comment on findings that is determined to be described for the one region of interest in the first determination in accordance with the plurality of pieces of new element information and present the rewritten comment on findings.

### Supplementary Note 12

The information processing apparatus according to any one of Supplementary Notes 1 to 11,
in which the processor is configured to:
   acquire a plurality of pieces of new element information about each of a plurality of new regions of interest for which a new comment on findings is to be created; and
   rewrite the comment on findings described for the regions of interest of which a number and a type match a number and a type of the plurality of new regions of interest, among the comments on findings that are determined to be described for the plurality of regions of interest in the first determination, in accordance with the plurality of pieces of new element information and present the rewritten comment on findings.

### Supplementary Note 13

The information processing apparatus according to any one of Supplementary Notes 10 to 12,
in which the new element information used for rewriting indicates at least one of a position, a measurement value, or the number of the new region of interest.

### Supplementary Note 14

The information processing apparatus according to any one of Supplementary Notes 10 to 13,
in which the processor is configured to receive an input of the new element information by a user.

### Supplementary Note 15

The information processing apparatus according to any one of Supplementary Notes 10 to 14,
in which the processor is configured to:
   acquire an image including at least one new region of interest for which a new comment on findings is to be created; and
   generate the new element information based on the image.

### Supplementary Note 16

The information processing apparatus according to any one of Supplementary Notes 1 to 15,
in which the element information includes at least one of a property, a position, a measurement value, a number, and a diagnosis of a region of interest, or a phrase expressing a change in the region of interest.

### Supplementary Note 17

An information processing method executed by a computer, the method comprising:
acquiring a comment on findings including a plurality of pieces of element information about at least one region of interest;
making a first determination as to whether the comment on findings is described for a plurality of regions of interest or for one region of interest based on the element information; and
determining a method for presenting the comment on findings based on a result of the first determination.

### Supplementary Note 18

An information processing program for causing a computer to execute a process comprising:
acquiring a comment on findings including a plurality of pieces of element information about at least one region of interest;
making a first determination as to whether the comment on findings is described for a plurality of regions of interest or for one region of interest based on the element information; and
determining a method for presenting the comment on findings based on a result of the first determination.

What is claimed is:

1. An information processing apparatus comprising a processor, wherein the processor is configured to:
acquire a comment on findings including a plurality of pieces of element information about at least one region of interest;
make a first determination as to whether the comment on findings is described for a plurality of regions of interest or for one region of interest based on the element information; and
determine a method for presenting the comment on findings based on a result of the first determination.

2. The information processing apparatus according to claim 1, wherein the processor is configured to, in a case of presenting at least one of a plurality of the comments on findings, lower a presentation priority of the comment on findings that is determined to be described for the plurality of regions of interest.

3. The information processing apparatus according to claim 1, wherein the processor is configured to:
make a second determination as to whether the comment on findings is described for a plurality of types of the regions of interest or for one type of the plurality of regions of interest, the comment on findings being determined to be described for the plurality of regions of interest in the first determination; and
determine the method for presenting the comment on findings based on a result of the second determination.

4. The information processing apparatus according to claim 3, wherein the processor is configured to, in a case of presenting at least one of a plurality of the comments on findings, lower a presentation priority of the comment on findings that is determined to be described for the plurality of types of the plurality of regions of interest.

5. The information processing apparatus according to claim 3, wherein the processor is configured to, in a case of presenting at least one of a plurality of the comments on findings, increase a presentation priority of the comment on findings described for the regions of interest of which a number and a type match a number and a type of new regions of interest for which a new comment on findings is to be created.

6. The information processing apparatus according to claim 1, wherein the processor is configured to make the first determination based on the number of pieces of element information indicating types of the regions of interest included in the comment on findings.

7. The information processing apparatus according to claim 1, wherein the processor is configured to, in a case in which the element information is divided into groups for each region of interest, make the first determination based on the number of groups.

8. The information processing apparatus according to claim 1, wherein:
the comment on findings consists of a plurality of partial sentences, and
the processor is configured to:
make the first determination for each of the partial sentences; and
determine a presentation method in a case in which the comment on findings is divided into the partial sentences and presented based on the result of the first determination.

9. The information processing apparatus according to claim 8, wherein the processor is configured to:
classify the element information included in the partial sentences into whether the element information is about an attention region of interest that is the region of interest being attended, whether the element information is about a non-attention region of interest that is the region of interest other than the attention region of interest, or whether the element information is common to the attention region of interest and the non-attention region of interest; and
determine that the partial sentence including the element information about the non-attention region of interest is described for the plurality of regions of interest.

10. The information processing apparatus according to claim 1, wherein the processor is configured to:
acquire new element information about one new region of interest for which a new comment on findings is to be created; and
rewrite the comment on findings that is determined to be described for the one region of interest in the first determination in accordance with the new element information and present the rewritten comment on findings.

11. The information processing apparatus according to claim 1, wherein the processor is configured to:
acquire a plurality of pieces of new element information about each of a plurality of new regions of interest for which a new comment on findings is to be created; and
rewrite the comment on findings that is determined to be described for the one region of interest in the first determination in accordance with the plurality of pieces of new element information and present the rewritten comment on findings.

12. The information processing apparatus according to claim 1, wherein the processor is configured to:
acquire a plurality of pieces of new element information about each of a plurality of new regions of interest for which a new comment on findings is to be created; and
rewrite the comment on findings described for the regions of interest of which a number and a type match a number and a type of the plurality of new regions of interest, among the comments on findings that are determined to be described for the plurality of regions of interest in the first determination, in accordance with the plurality of pieces of new element information and present the rewritten comment on findings.

13. The information processing apparatus according to claim 10, wherein the new element information used for rewriting indicates at least one of a position, a measurement value, or the number of the new region of interest.

14. The information processing apparatus according to claim 10, wherein the processor is configured to receive an input of the new element information by a user.

15. The information processing apparatus according to claim 10, wherein the processor is configured to:
acquire an image including at least one new region of interest for which a new comment on findings is to be created; and
generate the new element information based on the image.

16. The information processing apparatus according to claim 1, wherein the element information includes at least one of a property, a position, a measurement value, a number, and a diagnosis of a region of interest, or a phrase expressing a change in the region of interest.

17. An information processing method executed by a computer, the method comprising:
acquiring a comment on findings including a plurality of pieces of element information about at least one region of interest;
making a first determination as to whether the comment on findings is described for a plurality of regions of interest or for one region of interest based on the element information; and
determining a method for presenting the comment on findings based on a result of the first determination.

18. A non-transitory computer-readable storage medium storing an information processing program for causing a computer to execute a process comprising:
acquiring a comment on findings including a plurality of pieces of element information about at least one region of interest;
making a first determination as to whether the comment on findings is described for a plurality of regions of interest or for one region of interest based on the element information; and
determining a method for presenting the comment on findings based on a result of the first determination.
